(19) United States
(12) Patent Application Publication
(10) Pub. No.: US 2017/0354540 A1
(43) Pub. Date: Dec. 14, 2017

(54) LENS CLEANING SYSTEM FOR GOGGLES

(71) Applicant: Fox Head, Inc., Irvine, CA (US)

(72) Inventors: Fong S. Yang, Orange, CA (US); Nathan E. Heronen, Mission Viejo, CA (US)

(73) Assignee: Fox Head, Inc., Irvine, CA (US)

(21) Appl. No.: 15/182,018

(22) Filed: Jun. 14, 2016

(51) Int. Cl. A61F 9/02; B08B 1/00

(52) U.S. Cl. CPC A61F 9/029; B08B 1/006

(57) ABSTRACT

A goggle includes a frame, a lens disposed within the frame, and a continuous roll-off system coupled to at least one of the frame and the lens. The continuous roll-off system includes a first canister positioned at a first end of the goggle, a second canister positioned at an opposing second end of the goggle, and a continuous loop of film extending across the lens and configured to selectively cycle between the first canister and the second canister.

### BACKGROUND

The subject matter disclosed herein generally relates to a continuous lens cleaning system (e.g., a continuous roll-off system, a wiper system, etc.) configured to couple to or be integrated with eyewear and facilitate cleaning a lens of the eyewear.

Eyewear, such as goggles, may include a rip-off system and/or a roll-off system configured to facilitate removing debris from a lens hindering the vision of the wearer of the eyewear. However, such rip-off systems and roll-off systems are only effective as long as a supply of film of the respective system remains.

### SUMMARY

One embodiment relates to a goggle. The goggle includes a frame, a lens disposed within the frame, and a continuous roll-off system coupled to at least one of the frame and the lens. The continuous roll-off system includes a first canister positioned at a first end of the goggle, a second canister positioned at an opposing second end of the goggle, and a continuous loop of film extending across the lens and configured to cycle between the first canister and the second canister.

Another embodiment relates to a continuous roll-off system for goggles. The continuous roll-off system includes a first canister configured to couple to a first end of the goggles, a second canister configured to couple to an opposing second end of the goggles, and a continuous loop of film configured to extend across a lens of the goggles and selectively cycle between the first canister and the second canister.

Still another embodiment relates a goggle. The goggle includes a frame, a lens disposed within the frame, and a lens cleaning system coupled to at least one of the frame and the lens. The lens cleaning system includes a first canister positioned at a first end of the goggle, a second canister positioned at an opposing second end of the goggle, a cable extending between the first canister and the second canister, a cleaning element coupled to the cable and configured to remove particles from the lens, and an actuation mechanism configured to facilitate selectively cycling the cable between the first canister and the second canister such that the cleaning element selectively repositions across the lens from a first position to a second position.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The drawings are provided to illustrate example embodiments described herein and are not intended to limit the scope of the disclosure. Throughout the drawings, reference numbers may be re-used to indicate general correspondence between referenced elements.

FIG. 1 is a front perspective view of eyewear having a lens cleaning system, according to an exemplary embodiment;

FIG. 2 is an exploded, perspective view of the lens cleaning system of FIG. 1, according to an exemplary embodiment;

FIG. 3 is a bottom view of a first cover for a first canister of the lens cleaning system of FIG. 1, according to an exemplary embodiment;

FIG. 4 is a bottom view of a second cover for a second canister of the lens cleaning system of FIG. 1, according to an exemplary embodiment;

FIG. 5 is a front perspective view of eyewear having a lens cleaning system, according to another exemplary embodiment;

FIG. 6 is a detailed perspective view of a first canister of the lens cleaning system of FIG. 5, according to an exemplary embodiment;

FIG. 7 is a detailed perspective view of a second canister of the lens cleaning system of FIG. 5, according to an exemplary embodiment;

FIG. 8 is a detailed perspective view of a cleaning element of the lens cleaning system of FIG. 5, according to an exemplary embodiment; and

FIG. 9 is a detailed perspective view of a cleaning element of the lens cleaning system of FIG. 5, according to another exemplary embodiment.

### DETAILED DESCRIPTION

Various aspects of the disclosure will now be described with regard to certain examples and embodiments, which are intended to illustrate but not to limit the disclosure. Nothing in this disclosure is intended to imply that any particular feature or characteristic of the disclosed embodiments is essential. The scope of protection is defined by the claims that follow this description and not by any particular embodiment described herein. Before turning to the figures, which illustrate example embodiments in detail, it should be understood that the application is not limited to the details or methodology set forth in the description or illustrated in the figures. It should also be understood that the terminology is for the purpose of description only and should not be regarded as limiting.

Embodiments herein generally relate to a lens cleaning system (e.g., a continuous roll-off system, a wiper system, etc.) configured to facilitate cleaning a lens of eyewear (e.g., goggles, glasses, sunglasses, protective eyewear, etc.). The lens cleaning system may be configured to facilitate selectively cleaning the lens (e.g., removing debris from the lens hindering the vision of the wearer, etc.) in a relatively simple manner (e.g., easy, quick, etc.) and/or facilitate providing continuous cleaning (e.g., such that components of the lens cleaning system do not need to be refilled or replenished, etc.). Such eyewear having the lens cleaning system may be used in a number of activities, including without limitation: sports and athletics, including extreme sports such as motocross and snowmobiling; cycling activities, including auto racing, motorcycle riding and racing, BMX, mountain biking, etc.; with recreational vehicles including all-terrain vehicles (ATVs), dirt bikes, utility task vehicles (UTVs), snowmobiles, and other off-road vehicles; military applications; and/or construction applications to name just a few. Further details are provided herein.

According to an exemplary embodiment, the eyewear of the present disclosure includes a frame, a lens disposed within the frame, and a lens cleaning system coupled to the frame and/or the lens. Some traditional lens cleaning systems may include a non-continuous roll-off system having a roll of film that is selectively translated across the lens from one canister to another as the film becomes dirty (e.g., with dirt, mud, water, snow, etc.). Other traditional lens cleaning systems may include a rip-off system having a plurality of rip-off film elements that couple to the eyewear and may be torn or ripped off one-by-one as each becomes dirty. Such traditional lens cleaning systems only remain effective while there is a supply of film, such that once the roll of film or the rip-off film elements are spent, the wearer of the eyewear needs to replenish the lens cleaning system. Such replenishing of the supply of film may be disadvantageous, especially in competitive events where the wearer is unable to stop if the lens cleaning system is rendered unusable (e.g., which may lead to the lens becoming covered in mud, dirt, water, snow, etc. thereby hindering the vision of the wearer). Further, traditional lens cleaning systems may require that the eyewear includes a cylindrical lens that may cause distortion in the vision of the wearer (e.g., in a vertical plane, etc.).

According to an exemplary embodiment, the lens cleaning system of the present disclosure includes a continuous roll-off system having a continuous loop of film with a multi-stage debris removal system integrated into canisters. According to another exemplary embodiment, the lens cleaning system of the present disclosure includes a wiper system having a cable pulley system and a cleaning element (e.g., a wiper element, a squeegee element, etc.) that is translated across the lens by the cable pulley system. Advantageously, the continuous roll-off system and the wiper system eliminate the need to replenish film components (e.g., the roll-off film, the rip-off film elements, etc.) of traditional roll-off and rip-off systems. The wiper system may also advantageously facilitate the use of a toroidal lens and/or spherical lens. Such a toroidal and/or spherical lens may cause no or negligible distortion in the vision of the wearer in any direction.

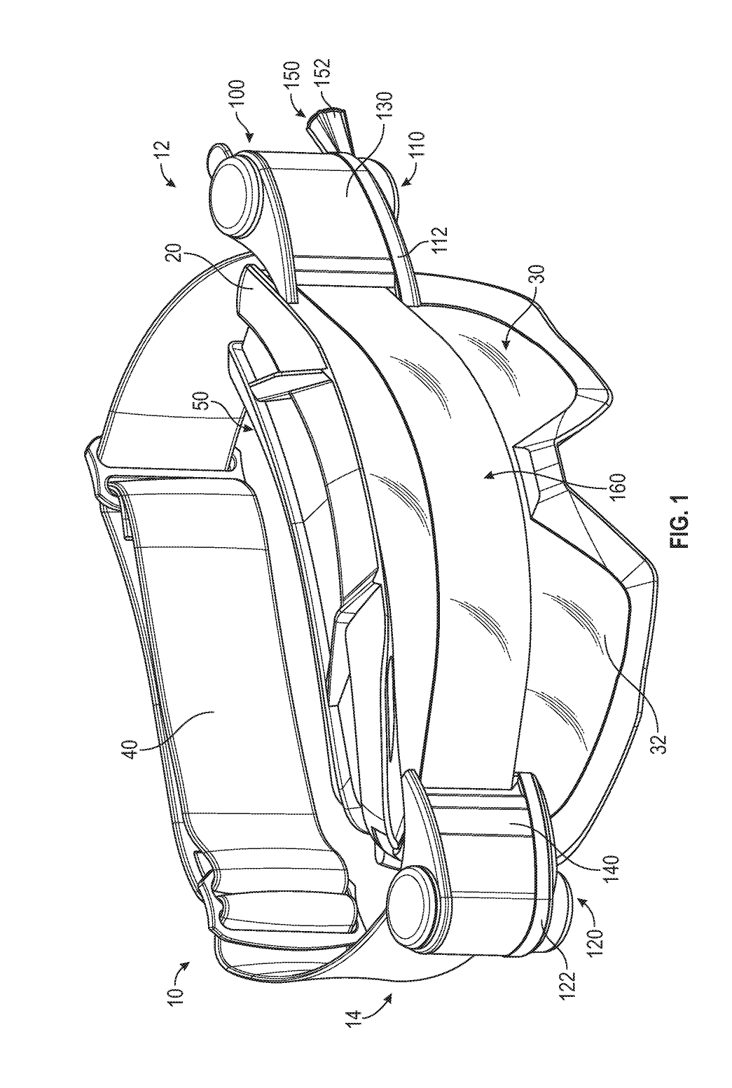

According to the exemplary embodiment shown in FIGS. 1-8, eyewear, shown as goggles 10, have a first lateral end, shown as right end 12, and an opposing second lateral end, shown as left end 14, and include a frame, shown as frame 20; a lens, shown as lens 30; a strap, shown as head strap 40; a cushion member, shown as compressible member 50; and one of (i) a first lens cleaning system, shown as continuous roll-off system 100, and (ii) a second lens cleaning system, shown as wiper system 200. According to an exemplary embodiment, the continuous roll-off system 100 and the wiper system 200 are configured to facilitate selectively cleaning the lens 30 (e.g., removing debris from the lens 30 hindering the vision of the wearer, etc.) in a relatively simple manner (e.g., easy, quick, etc.), while providing continuous cleaning (e.g., a film component of the continuous roll-off system 100 does not need to be refilled or replenished, the wiper system 200 does not include a film component, etc.). According to an exemplary embodiment, the eyewear are motocross goggles. In other embodiments, the eyewear are snowmobiling goggles, snowboarding goggles, mountain biking goggles, motorcycle goggles, sky diving goggles, or still another action or extreme sport goggles. In still other embodiments, the eyewear are or include swimming goggles or other sports goggles (e.g., used in basketball, baseball, etc.; Rec Specs®; etc.). In another embodiment, the eyewear is or includes a visor coupled to a helmet (e.g., a football helmet, a motorcycle helmet, etc.). In yet other embodiments, the eyewear are another type of eyewear used for construction, military applications, machining, carpentry, scientific experimentation, and/or the like. In still other embodiments, the eyewear are traditional vision enhancing glasses (e.g., prescription glasses, etc.) and/or sunglasses.

According to an exemplary embodiment, the goggles 10 provide protection to the eyes and adjacent area of the face of a wearer. The lens 30 and/or the frame 20 may intercept light, wind, rain, snow, water, particulate matter (e.g., dust, dirt, mud, etc.), and the like to protect the eyes and/or various portions of the face of a wearer of the goggles 10. According to an exemplary embodiment, the head strap 40 includes a band (e.g., an elastic band, etc.) having length adjuster elements. The length adjuster elements may be configured to facilitate adjusting (e.g., extending, shortening, etc.) the size of the head strap 40 such that the goggles 10 may fit various sized heads.

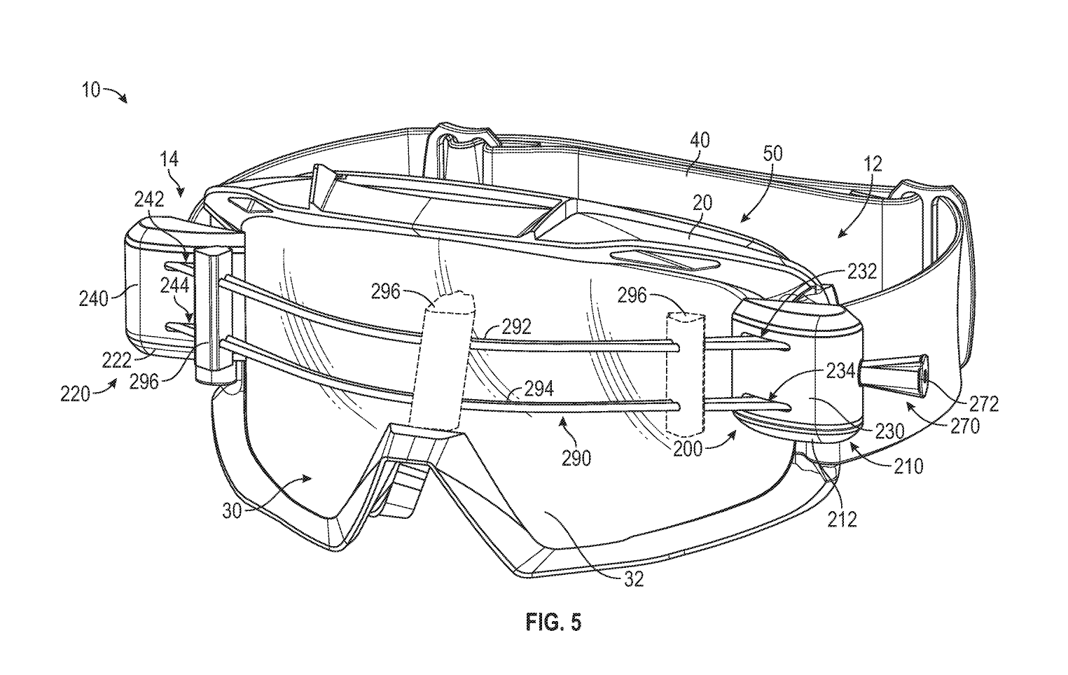

According to an exemplary embodiment, the frame 20 is shaped to correspond with anatomical features of a wearer's face (e.g., cheeks, nose, forehead, etc.) to facilitate a proper fit of the goggles 10 when worn. As shown in FIGS. 1 and 5, the compressible member 50 is positioned along an interior of the frame 20 such that the compressible member 50 is in contact with a wearer's face when the goggles 10 are worn. The compressible member 50 may include an impact attenuating material and/or cushion material (e.g., expanded polyurethane (EPU) foam, expanded polypropylene (EPP) foam, expanded polyethylene (EPE) foam, polyolefin foam, etc.) to facilitate a snug fit and comfortable fit of the goggles 10 onto a wearer's face and/or mitigate impact forces encountered by the goggles 10 (e.g., from flying debris, etc.).

According to an exemplary embodiment, the frame 20 defines a cavity or opening configured receive the lens 30 such that the lens 30 is disposed across and/or within, and supported by the frame 20. As shown in FIGS. 1 and 5, the lens 30 includes a unitary, arcuate lens that extends across the entire opening of the frame 20 (e.g., over both the left and right eyes of the wearer, from the right end 12 to the left end 14, etc.). In an alternative embodiment, the lens 30 includes dual lenses, one positioned over each of the left and right eyes of the wearer. In some embodiments, the lens 30 is clear (e.g., substantially transparent, etc.). In other embodiments, the lens 30 includes a tinted and/or polarized coating to shade a wearer's eyes from sunlight, reduce glare, improve contrast, and/or enhance depth perception. The tinted and/or polarized coating may be various colors (e.g., black, yellow, blue, green, brown, gray, red, etc.). In some embodiments, the lens 30 includes a reflective coating (e.g., to prevent others from seeing where the wearer is looking, etc.). In some embodiments, the lens 30 is a prescription lens configured to enhance the vision of a wearer of the goggles 10. In some embodiments, the lens 30 is or includes at least one of a toroidal lens and a spherical lens (e.g., when the lens cleansing system includes the wiper system 200, preventing distortion in the vision of the wearer, etc.). In some embodiments, the lens 30 is a cylindrical lens. In other embodiments, the lens 30 is still otherwise shaped.

### Continuous Roll-Off System

As shown in FIG. 1, the continuous roll-off system 100 includes a first canister, shown as right canister 110, positioned at the right end 12 of the goggles 10; a second canister, shown as left canister 120, positioned at the left end 14 of the goggles 10; an actuation mechanism, shown as actuation system 150; and a continuous loop of film, shown as continuous film 160. In some embodiments, the right canister 110 and/or the left canister 120 are coupled (e.g., attached, releasably secured, fixed, etc.) to the lens 30. In other embodiments, the right canister 110 and/or the left canister 120 are coupled (e.g., attached, releasably secured, fixed, etc.) to the frame 20. As shown in FIG. 1, the continuous film 160 extends across a surface of the lens 30, shown as front surface 32, between the right canister 110 and the left canister 120. According to an exemplary embodiment, the actuation system 150 is configured to facilitate selectively cycling the continuous film 160 across the front surface 32 of the lens 30 between the right canister 110 and the left canister 120.

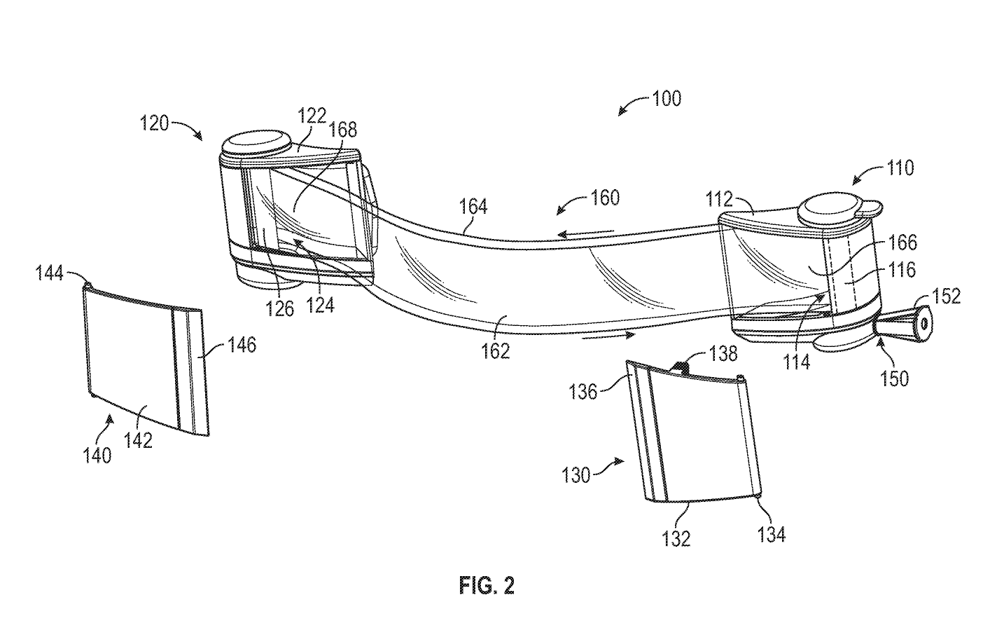

As shown in FIGS. 1-2, the right canister 110 includes a first housing, shown as right housing 112, a first spindle, shown as right spindle 116, and a first cap, shown as right cover 130. As shown in FIG. 2, the right housing 112 defines a first interior, shown as right cavity 114, configured to receive the right spindle 116. As shown in FIGS. 1-2, the right cover 130 is configured to enclose the right cavity 114 of the right canister 110.

As shown in FIGS. 1-2, the left canister 120 includes a second housing, shown as left housing 122, a second spindle, shown as left spindle 126, and a second cap, shown as left cover 140. As shown in FIG. 2, the left housing 122 defines a second interior, shown as left cavity 124, configured to receive the left spindle 126. As shown in FIGS. 1-2, the left cover 140 is configured to enclose the left cavity 124 of the left canister 120.

As shown in FIG. 2, the continuous film 160 includes a first portion, shown as front portion 162, a second portion, shown as rear portion 164, a first end portion, shown as right portion 166, and an opposing second end portion, shown as left portion 168. According to an exemplary embodiment, the front portion 162, the rear portion 164, the right portion 166, and the left portion 168 form a single, continuous loop of film. As shown in FIG. 2, the right portion 166 of the continuous film 160 extends within the right cavity 114 of the right canister 110 and around the right spindle 116, and the left portion 168 of the continuous film 160 extends within the left cavity 124 of the left canister 120 and around the left spindle 126. According to an exemplary embodiment, the rear portion 164 of the continuous film 160 is configured to be disposed along the front surface 32 of the lens 30 and the front portion 162 of the continuous film 160 is configured to be disposed along the rear portion 164.

As shown in FIGS. 1-2, the actuation system 150 is coupled to the right housing 112 of the right canister 110 and includes a pull-cord, shown as pull-string 152. According to an exemplary embodiment, the pull-string 152 is coupled to the right spindle 116 such that pulling the pull-string 152 rotates the right spindle 116 and facilitate manually cycling the continuous film 160 between the right canister 110 and the left canister 120. The actuation system 150 may include a rotational biasing member (e.g., a torsion/rotational spring, etc.) that winds the pull-string 152 up into the right canister 110 (e.g., in response to the user releasing the pulled pull-string 152, etc.). In other embodiments, the actuation system 150 is coupled to the left housing 122 of the left canister 120 such that pulling the pull-string 152 rotates the left spindle 126 and facilitate manually cycling the continuous film 160 between the left canister 120 and the right canister 110. In an alternative embodiment, the actuation system 150 additionally or alternatively includes an electric actuator positioned to rotate at least one of the right spindle 116 and the left spindle 126 (e.g., in response to wearer pressing a rotate button, etc.) to selectively cycle the continuous film 160 between the right canister 110 and the left canister 120.

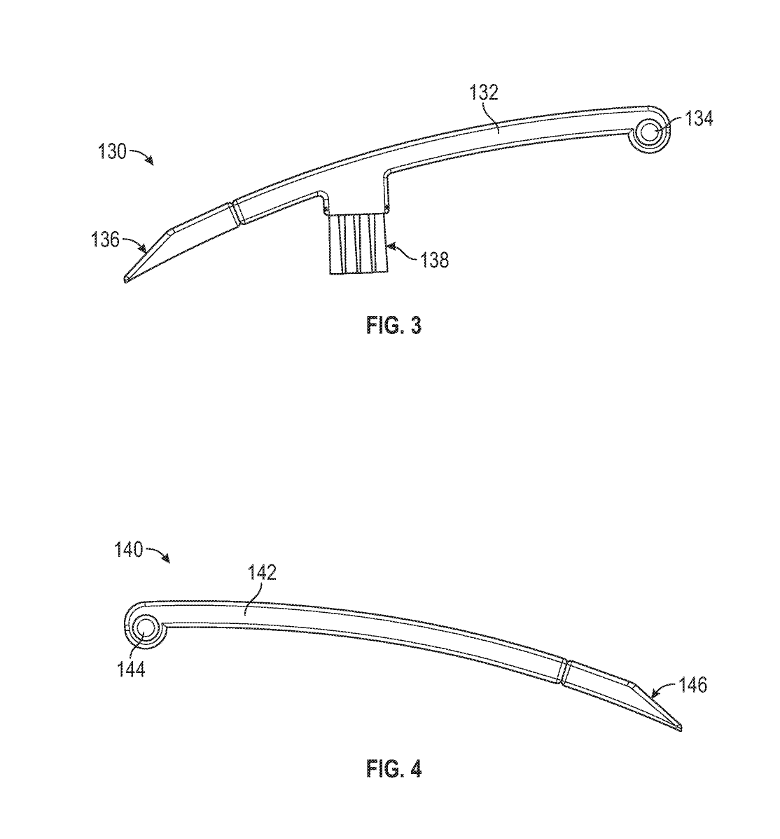

As shown in FIGS. 2-3, the right cover 130 has a first body, shown as right plate 132, that includes a first interface, shown as right coupler 134. According to an exemplary embodiment, the right coupler 134 is configured to couple (e.g., rotatably couple, etc.) the right cover 130 to the right housing 112. As shown in FIGS. 2-3, the right plate 132 includes a leading or front edge having a first scraper element, shown as right scraper 136. According to an exemplary embodiment, the right scraper 136 is positioned to engage the front portion 162 of the continuous film 160 to remove particles (e.g., debris, mud, dirt, larger particles, etc.) from the front portion 162 as the continuous film 160 enters the right canister 110. As shown in FIGS. 2-3, the right plate 132 includes a cleaning element, shown as cleaner 138, extending from an interior of the right plate 132. According to an exemplary embodiment, the cleaner 138 is positioned within the right cavity 114 of the right canister 110 to engage the right portion 166 of the continuous film 160. The cleaner 138 may be configured to remove finer particles from the continuous film 160 and/or dry the continuous film 160 as the continuous film 160 cycles. In some embodiments, the cleaner 138 includes a drying element (e.g., a squeegee, an absorbent pad, a microfiber cloth, etc.) configured to dry liquid (e.g., water, snow, rain, etc.) that may be disposed on the continuous film 160. In some embodiments, the cleaner 138 includes a scraper element or brush element configured to remove finer particles that may have passed by the right scraper 136.

As shown in FIGS. 2 and 4, the left cover 140 has a second body, shown as left plate 142, that includes a second interface, shown as left coupler 144. According to an exemplary embodiment, the left coupler 144 is configured to couple (e.g., rotatably couple, etc.) the left cover 140 to the left housing 122. As shown in FIGS. 2 and 4, the left plate 142 includes a leading or front edge having a second scraper element, shown as left scraper 146. According to an exemplary embodiment, the left scraper 146 is positioned to engage the front portion 162 of the continuous film 160 to remove particles (e.g., debris, mud, dirt, particles that may have cycled past the right canister 110, etc.) from the front portion 162 as the continuous film 160 exits the left canister 120. In some embodiments, the left plate 142 additionally or alternatively includes a cleaning element (e.g., similar to the cleaner 138, etc.) extending from an interior of the left plate 142. The cleaner may be positioned within the left cavity 124 of the left canister 120 to engage the left portion 168 of the continuous film 160. The cleaner may be configured to remove finer particles from the continuous film 160 and/or dry the continuous film 160 as the continuous film 160 cycles. According to an exemplary embodiment, the right scraper 136, the cleaner 138, and/or the left scraper 146 form a multi-stage debris removal system of the continuous roll-off system 100. Such a multi-stage debris removal system may facilitate the use of the continuous film 160 (e.g., such that roll-off film of traditional roll-off systems does not need to be used, thereby preventing the need for replacing a film component, etc.).

### Wiper System

As shown in FIG. 5, the wiper system 200 includes a first canister, shown as right canister 210, positioned at the right end 12 of the goggles 10; a second canister, shown as left canister 220, positioned at the left end 14 of the goggles 10; an actuation mechanism, shown as actuation system 270; and a cable system, shown as pulley system 290. In some embodiments, the right canister 210 and/or the left canister 220 are coupled (e.g., attached, releasably secured, fixed, etc.) to the lens 30. In other embodiments, the right canister 210 and/or the left canister 220 are coupled (e.g., attached, releasably secured, fixed, etc.) to the frame 20.

As shown in FIGS. 5 and 7, the right canister 210 includes a first housing, shown as right housing 212, a first cap, shown as right cover 230, and a first spindle, shown as right spindle 250. As shown in FIG. 7, the right housing 212 defines a first interior, shown as right cavity 214, configured to receive the right spindle 250. The right spindle 250 has a first portion, shown as upper cable portion 252, a second portion, shown as pull-cord portion 254, and a third portion, shown as lower cable portion 256. As shown in FIGS. 5 and 7, the right cover 230 defines a first aperture, shown as upper cable slot 232, positioned to align with the upper cable portion 252 of the right spindle 250, a second aperture, shown as lower cable slot 234, positioned to align with the lower cable portion 256 of the right spindle 250, and a third aperture, shown as pull-cord slot 236, positioned to align with the pull-cord portion 254 of the right spindle 250.

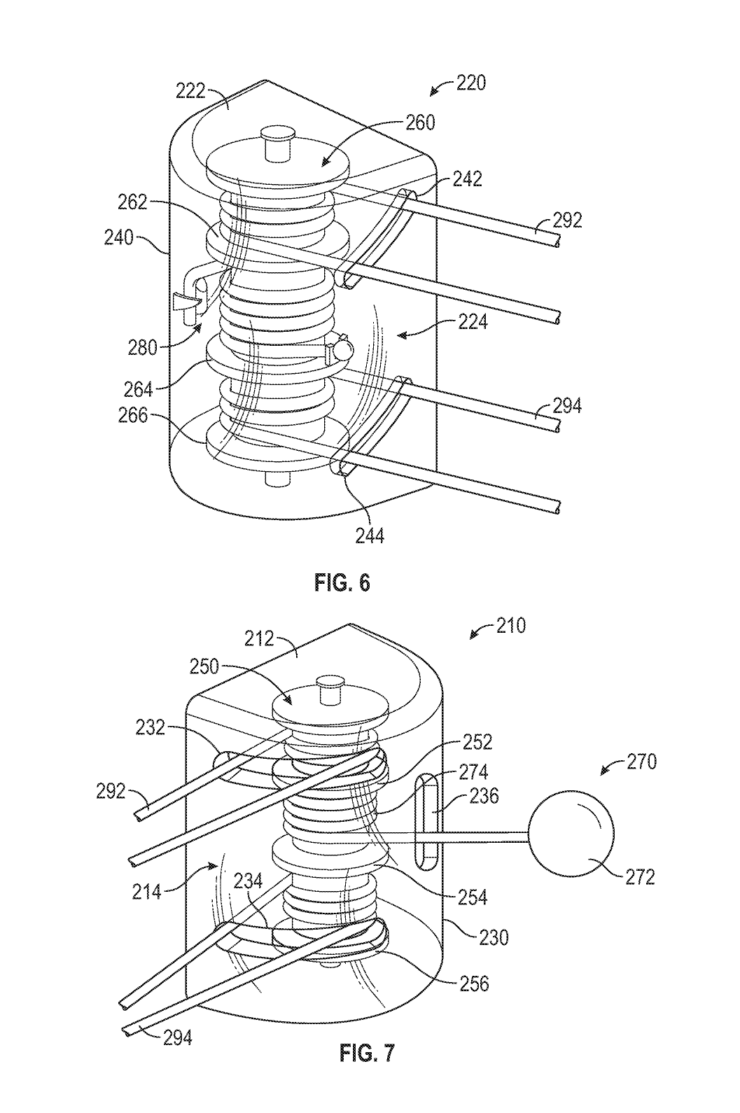

As shown in FIGS. 5-6, the left canister 220 includes a second housing, shown as left housing 222, a second cap, shown as left cover 240, and a second spindle, shown as left spindle 260. As shown in FIG. 6, the left housing 222 defines a second interior, shown as left cavity 224, configured to receive the left spindle 260. The left spindle 260 has a first portion, shown as upper cable portion 262, a second portion, shown as biasing portion 264, and a third portion, shown as lower cable portion 266. As shown in FIGS. 5-6, the left cover 240 defines a first aperture, shown as upper cable slot 242, positioned to align with the upper cable portion 262 of the left spindle 260 and a second aperture, shown as lower cable slot 244, positioned to align with the lower cable portion 266 of the left spindle 260.

As shown in FIG. 5, the pulley system 290 includes a first cable (e.g., a first monofilament cable pulley, etc.), shown upper cable 292, a second cable (e.g., a second monofilament cable pulley, etc.), shown as lower cable 294, and a cleaning element, shown as squeegee 296, coupled to the upper cable 292 and the lower cable 294. In other embodiments, the pulley system 290 includes a different number of cables (e.g., one cable, three cables, etc.). As shown in FIG. 5, the upper cable 292 and the lower cable 294 extend between the right canister 210 and the left canister 220.

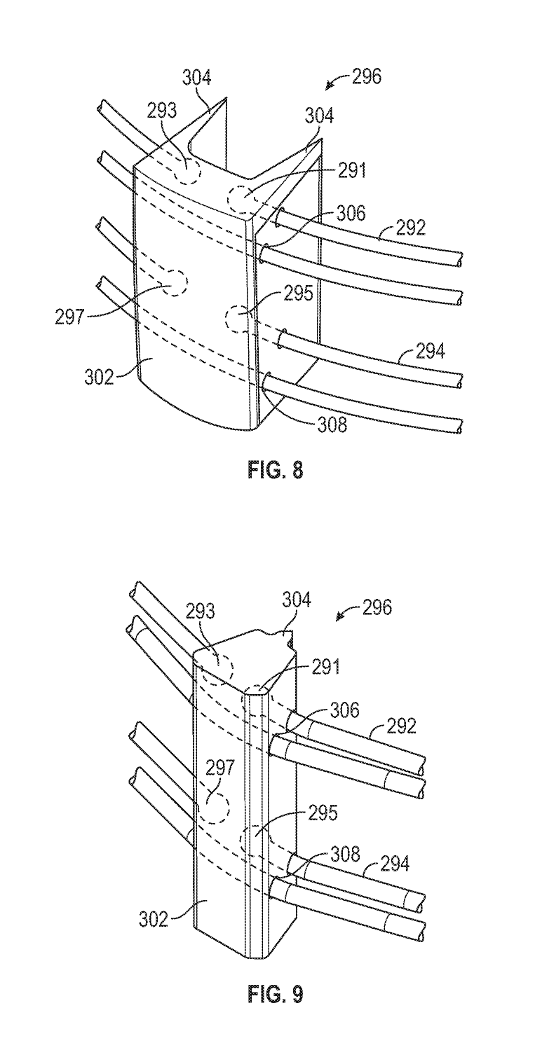

As shown in FIGS. 8-9, the squeegee 296 includes a body portion, shown as body 302, having at least one protrusion, shown as wiper blades 304, extending therefrom. As shown in FIG. 9, the squeegee 296 includes one wiper blade 304. As shown in FIG. 8, the squeegee 296 includes a pair of wiper blades 304. In other embodiments, the squeegee 296 includes greater than two wiper blades 304 (e.g., three, four, etc.). The wiper blades 304 of the squeegee 296 may have a complimentary shape to the front surface 32 of the lens 30. By way of example, the wiper blades 304 may have a shape that corresponds with a toroidal lens, a spherical lens, a cylindrical lens, and/or another shaped lens. According to an exemplary embodiment, the wiper blades 304 are configured to engage the front surface 32 of the lens 30 and remove particles (e.g., dirt, mud, water, snow, etc. by scraping/wiping across the front surface 32, etc.) therefrom as the squeegee 296 is selectively repositioned across the lens 30 from a first position (e.g., proximate the left end 14, etc.) to a second position (e.g., proximate the right end 12, etc.). Having two or more wiper blades 304 may provide greater stability (e.g., prevent the squeegee 296 from pivoting about a single wiper blade 304, etc.) as the squeegee 296 is selectively repositioned across the lens 30.

As shown in FIGS. 8-9, the upper cable 292 includes a first end, shown as first end 291, and an opposing second end, shown as second end 293. The first end 291 is coupled to (e.g., attached to, embedded within, hard mounted to, etc.) a first side (e.g., a right side, etc.) of the body 302 of the squeegee 296 and the second end 293 is coupled to (e.g., attached to, embedded within, hard mounted to, etc.) an opposing second side (e.g., a left side, etc.) of the body 302 of the squeegee 296. As shown in FIGS. 5-9, the upper cable 292 (e.g., the first end 291 thereof, etc.) extends from the first side of the squeegee 296, enters the right canister 210 through the upper cable slot 232, coils around the upper cable portion 252 of the right spindle 250, exits the right canister 210 through the upper cable slot 232, enters a first aperture, shown as upper through-hole 306, defined by body 302 of the squeegee 296 through the first side of the body 302, exits the upper through-hole 306 through the opposing second side of the body 302, enters the left canister 220 through the upper cable slot 242, coils around the upper cable portion 262 of the left spindle 260, exits the left canister 220 through the upper cable slot 242, and terminates within the second side of the squeegee 296 (e.g., the second end 293 thereof, etc.). The upper cable 292 may thereby forms a single, continuous cable (e.g., a monofilament cable, etc.) that extends around the right spindle 250 and the left spindle 260, coupling the squeegee 296 thereto. In other embodiments, the upper cable 292 includes two or more portions or cables. By way of example, a first upper cable may have both ends thereof embedded within the first side of the body 302 of the squeegee 296 and extend around the upper cable portion 252 of the right spindle 250 and a second upper cable may have both ends thereof embedded within the opposing second side of the body 302 of the squeegee 296 and extend around the upper cable portion 262 of the left spindle 260.

As shown in FIGS. 8-9, the lower cable 294 includes a first end, shown as first end 295, and an opposing second end, shown as second end 297. The first end 295 is coupled to (e.g., attached to, embedded within, hard mounted to, etc.) the first side (e.g., the right side, etc.) of the body 302 of the squeegee 296 and the second end 293 is coupled to (e.g., attached to, embedded within, hard mounted to, etc.) the opposing second side (e.g., the left side, etc.) of the body 302 of the squeegee 296. As shown in FIGS. 5-9, the lower cable 294 (e.g., the first end 295 thereof, etc.) extends from the first side of the squeegee 296, enters the right canister 210 through the lower cable slot 234, coils around the lower cable portion 256 of the right spindle 250, exits the right canister 210 through the lower cable slot 234, enters a second aperture, shown as lower through-hole 308, defined by body 302 of the squeegee 296 through the first side of the body 302, exits the lower through-hole 308 through the opposing second side of the body 302, enters the left canister 220 through the lower cable slot 244, coils around the lower cable portion 266 of the left spindle 260, exits the left canister 220 through the lower cable slot 244, and terminates within the second side of the squeegee 296 (e.g., the second end 297 thereof, etc.). The lower cable 294 may thereby forms a single, continuous cable (e.g., a monofilament cable, etc.) that extends around the right spindle 250 and the left spindle 260, coupling the squeegee 296 thereto. In other embodiments, the lower cable 294 includes two or more portions or cables. By way of example, a first lower cable may have both ends thereof embedded within the first side of the body 302 of the squeegee 296 and extend around the lower cable portion 256 of the right spindle 250 and a second lower cable may have both ends thereof embedded within the opposing second side of the body 302 of the squeegee 296 and extend around the lower cable portion 266 of the left spindle 260.

As shown in FIGS. 5 and 7, the actuation system 270 is coupled to the right canister 210 and includes a pull-cord including a pull-string 274 with a knob 272 attached to an end thereof. In other embodiments, the actuation system 270 is coupled to the left canister 220. According to an exemplary embodiment, the actuation system 270 is configured to facilitate selectively cycling the upper cable 292 and the lower cable 294 between the right canister 210 and the left canister 220 such that the squeegee 296 selectively repositions across the front surface 32 of the lens 30 from the first position to the second position. As shown in FIG. 7, the pull-string 274 is coupled to (e.g., coiled around, etc.) the pull-cord portion 254 of the right spindle 250 with the end thereof extending through the pull-cord slot 236 such that the knob 272 is positioned outside of the right canister 210. According to an exemplary embodiment, pulling the knob 272 causes the pull-string 274 to unwind from the right spindle 250, thereby rotating the right spindle 250. Such rotation of the right spindle 250 may cause the upper cable 292 and the lower cable 294 to selectively cycle between the right canister 210 and the left canister 220 such that the left spindle 260 also rotates, thereby causing the squeegee 296 to selectively reposition across the lens 30.

As shown in FIG. 6, the wiper system 200 includes a resilient member, shown as rotational biasing element 280 (e.g., a torsional spring, a rotational spring, etc.), coupled to the left housing 222 (e.g., an end thereof attached to the left housing 222, etc.) and the biasing portion 264 of the left spindle 260 (e.g., coiled around the biasing portion 264, etc.). The rotational biasing element 280 may provide a biasing force that rotationally biases the left spindle 260 into a nominal position such that when the knob 272 is released, the squeegee 296 returns to the first position (e.g., the left end 14, etc.). In other embodiments, the rotational biasing element 280 is coupled to the right spindle 250. In still other embodiments, the wiper system 200 does not include the rotational biasing element 280 (e.g., in embodiments with an electric actuator, etc.). By way of example, the actuation system 270 may additionally or alternatively include an electric actuator positioned to rotate at least one of the right spindle 250 and the left spindle 260 (e.g., in response to wearer pressing a rotate button, etc.) to selectively rotate the spindles and thereby reposition the squeegee 296 across the lens 30.

It is important to note that the construction and arrangement of the elements of the systems, methods, and/or apparatuses as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the enclosure may be constructed from any of a wide variety of materials that provide sufficient strength or durability, in any of a wide variety of colors, textures, and combinations.

Embodiments have been described in connection with the accompanying drawings. However, it should be understood that the figures are not drawn to scale. Distances, angles, shapes, etc. are merely illustrative and do not necessarily bear an exact relationship to actual dimensions and layout of the articles that are illustrated. In addition, the foregoing embodiments have been described at a level of detail to allow one of ordinary skill in the art to make and use the articles, parts, different materials, etc. described herein. A wide variety of variation is possible. Articles, materials, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or configurations are in any way required for one or more embodiments. The terms “comprising,” “including,” “having,” and the like are synonymous and are used inclusively, in an open-ended fashion, and do not exclude additional elements, features, acts, operations, and so forth. The term “consisting essentially of” can be used anywhere where the terms comprising, including, containing or having are used herein, but consistent essentially of is intended to mean that the claim scope covers or is limited to the specified materials or steps recited and those that do not materially affect the basic and novel characteristic(s) of the claimed invention. Also, the term “consisting of” can be used anywhere where the terms comprising, including, containing or having are used herein, but consistent of excludes any element, step, or ingredient not specified in a given claim where it is used.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

Additionally, in the subject description, the word “exemplary” is used to mean serving as an example, instance, or illustration. Any embodiment or design described herein as “exemplary” is not necessarily to be construed as preferred or advantageous over other embodiments or designs. Rather, use of the word exemplary is intended to present concepts in a concrete manner. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A goggle, comprising:
a frame;
a lens disposed within the frame; and
a continuous roll-off system coupled to at least one of the frame and the lens, the continuous roll-off system including:
a first canister positioned at a first end of the goggle;
a second canister positioned at an opposing second end of the goggle; and
a continuous loop of film extending across the lens and configured to selectively cycle between the first canister and the second canister.

2. The goggle of claim 1, wherein the first canister includes a first spindle positioned within a first interior of the first canister and the second canister includes a second spindle positioned within a second interior of the second canister, and wherein the continuous loop of film forms a single loop that extends around the first spindle and the second spindle.

3. The goggle of claim 2, wherein the first canister includes a cover that encloses the first interior of the first canister.

4. The goggle of claim 3, wherein the cover includes a scraper element positioned to remove particles from the continuous loop of film as the continuous loop of film enters the first canister.

5. The goggle of claim 4, wherein the cover includes a cleaning element positioned to engage with the continuous loop of film within the first canister.

6. The goggle of claim 2, wherein the continuous roll-off system further includes an actuation mechanism configured to rotate at least one of the first spindle and the second spindle to selectively cycle the continuous loop of film between the first canister and the second canister.

7. The goggle of claim 6, wherein the actuation mechanism includes a pull-cord configured to facilitate manually cycling the continuous loop of film between the first canister and the second canister.

8. A roll-off system for goggles, comprising:
a first canister configured to couple to a first end of the goggles;
a second canister configured to couple to an opposing second end of the goggles; and
a continuous loop of film configured to extend across a lens of the goggles and selectively cycle between the first canister and the second canister.

9. The roll-off system of claim 8, wherein the first canister includes a scraper element positioned to remove particles from the continuous loop of film as the continuous loop of film enters the first canister.

10. The roll-off system of claim 8, wherein the first canister includes a cleaning element positioned within the first canister to engage with the continuous loop of film.

11. The roll-off system of claim 8, wherein the first canister includes a first spindle positioned within a first interior of the first canister and the second canister includes a second spindle positioned within a second interior of the second canister, and wherein the continuous loop of film forms a single loop that extends around the first spindle and the second spindle.

12. The roll-off system of claim 11, further comprising an actuation mechanism configured to rotate at least one of the first spindle and the second spindle to selectively cycle the continuous loop of film between the first canister and the second canister.

13. The roll-off system of claim 12, wherein the actuation mechanism includes a pull-cord configured to facilitate manually cycling the continuous loop of film between the first canister and the second canister.

14. A goggle, comprising:
a frame;
a lens disposed within the frame; and
a lens cleaning system coupled to at least one of the frame and the lens, the lens cleaning system including:
a first canister positioned at a first end of the goggle;
a second canister positioned at an opposing second end of the goggle;
a cable extending between the first canister and the second canister;
a cleaning element coupled to the cable, the cleaning element configured to remove particles from the lens; and
an actuation mechanism configured to facilitate selectively cycling the cable between the first canister and the second canister such that the cleaning element selectively repositions across the lens from a first position to a second position.

15. The goggle of claim 14, wherein the first canister includes a first spindle and the second canister includes a second spindle, and wherein the cable extends around the first spindle and the second spindle.

16. The goggle of claim 15, wherein the actuation mechanism include a pull-cord coupled to the first spindle, the pull-cord configured to facilitate manually cycling the cable between the first canister and the second canister such that the cleaning element selectively repositions across the lens.

17. The goggle of claim 16, further comprising a resilient member coupled to the second spindle, the resilient member configured to provide a biasing force that returns the cleaning element to the first position.

18. The goggle of claim 14, wherein the cleaning element includes a squeegee having at least one blade configured to scrape the particles from the lens as the cleaning element is selectively repositioned.

19. The goggle of claim 18, wherein the squeegee has a pair of blades.

20. The goggle of claim 14, wherein the lens includes at least one of a toroidal lens portion and a spherical lens portion, and wherein the cleaning element has a complimentary shape to the at least one of the toroidal lens portion and the spherical lens portion.
